(19) United States
(12) Patent Application Publication
(10) Pub. No.: US 2026/0034328 A1
(43) Pub. Date: Feb. 5, 2026

(54) RESPIRATORY MASK FOR SUPPLYING A PATIENT WITH RESPIRATORY GAS

(71) Applicant: Loewenstein Medical Technology S.A., Luxembourg (LU)

(72) Inventor: Martin EIFLER, Glueckstadt (DE)

(21) Appl. No.: 19/276,590

(22) Filed: Jul. 22, 2025

(30) Foreign Application Priority Data
Jul. 30, 2024 (DE) 10 2024 121 700.6

(51) Int. Cl. A61M 16/06

(52) U.S. Cl. CPC A61M 16/0694

(57) ABSTRACT

A respiratory mask for supplying a patient with respiratory gas comprises a mask body for applying to mouth and/or nose of the patient, comprising a first and a second coupling receptacle and a tube connector for attachment of a breathing tube; a head harness for fixing the mask body with respect to the patient's face and comprising a first and a second strap for tensioning around the patient's head; a first and a second coupling element for coupling the head harness to the first and second coupling receptacle. Each coupling element comprises a coupling portion for coupling the coupling element to the respective coupling receptacle and a first and second strap receptacle for fastening the first and second strap and is configured in a particular way as set forth in the claims.

### CROSS-REFERENCE TO RELATED APPLICATIONS

The present application claims priority under 35 U.S.C. § 119 of German Patent Application No. 10 2024 121 700.6, filed Jul. 30, 2024, the entire disclosure of which is expressly incorporated by reference herein.

### BACKGROUND OF THE INVENTION

### 1. Field of the Invention

The invention relates to a respiratory mask for supplying a patient with respiratory gas.

### 2. Discussion of Background Information

Respiratory masks, used as an interface between a patient and a ventilator, must meet high standards of stability, safety and comfort while at the same time being easy to handle. Such respiratory masks are usually fixed to the patient's head by one or more straps. Especially during home ventilation, the patient is often responsible for the correct application and fixing of the respiratory mask on the face.

Some patients, for example those with limited mobility, the elderly or children, may find it difficult to apply the respiratory mask. However, a prerequisite for safe and efficient ventilation is that the respiratory mask is correctly fastened and sits in a stable position and with as close as possible a fit.

In view of the foregoing, it would be advantageous to have available a respiratory mask which is particularly easy to apply or remove and/or which affords improved wearing comfort.

### SUMMARY OF THE INVENTION

In a first aspect, the invention provides a respiratory mask for supplying a patient with respiratory gas. The respiratory mask comprises: a mask body for applying to the mouth and/or nose of the patient, wherein the mask body comprises a first coupling receptacle, a second coupling receptacle, and a tube connector for the attachment of a breathing tube; a head harness for fixing the mask body with respect to the patient's face, wherein the head harness comprises a first strap and a second strap for tensioning around the patient's head; a first coupling element for coupling the head harness to the first coupling receptacle; a second coupling element for coupling the head harness to the second coupling receptacle. Each coupling element comprises a coupling portion for coupling the coupling element to the respective coupling receptacle, a first strap receptacle for fastening the first strap, and a second strap receptacle for fastening the second strap. Each coupling element is configured such that, when the mask body is fixed with respect to the patient's face by means of the head harness, the first strap receptacle, viewed in the direction of a sagittal axis of the patient, is arranged at least partially between the chin and an ear of the patient and, viewed in the direction of a longitudinal axis of the patient orthogonal to the sagittal axis, is arranged at least partially between the chin and the temporal region of the patient, and the second strap receptacle is arranged at least partially opposite the temporal region of the patient.

On account of the special arrangement of the strap receptacles with respect to the chin, ears and temporal region of the patient, such a respiratory mask is particularly easy to apply or remove and particularly comfortable to wear. In addition, on account of the resulting special lever ratios, the respiratory mask ensures a secure and sufficiently tight fit of the mask body. In particular, the respiratory mask allows the coupling elements and/or straps (or at least a large part of them) to be positioned outside the patient's line of sight or field of view, which is a benefit for spectacle wearers for example. Another advantage is that the respiratory mask does not require a potentially annoying forehead support, which fact further simplifies the handling of the respiratory mask.

In a second aspect the invention provides a respiratory mask for supplying a patient with respiratory gas. The respiratory mask comprises: a mask body for applying to the mouth and/or nose of the patient, wherein the mask body comprises a first coupling receptacle, a second coupling receptacle, and a tube connector for the attachment of a breathing tube; a head harness for fixing the mask body with respect to the patient's face; a first coupling element for coupling the head harness to the first coupling receptacle; a second coupling element for coupling the head harness to the second coupling receptacle. Each coupling element comprises a coupling portion for coupling the coupling element to the respective coupling receptacle, and one or more strap receptacles for fastening one or more straps of the head harness. The coupling portion of at least one (or each) of the two coupling elements is connectable to the respective coupling receptacle to form a plug connection. Furthermore, the respiratory mask comprises a locking element movably mounted on the mask body and/or on the respective coupling element between a locking position and an unlocking position, which locking element is configured to lock the plug connection in the locking position, so that a movement of the coupling portion in a separation direction away from the respective coupling receptacle is not possible, and to unlock it in the unlocking position (such that the coupling portion can be moved in the separation direction). In addition, the respiratory mask comprises an actuation element movably mounted on the mask body and/or on the respective coupling element between a rest position and an actuation position, for actuating the locking element when the plug connection is locked. The actuation element is configured to press against the locking element when moving into the actuation position, such that the locking element is moved into the unlocking position.

On account of the plug connection, such a respiratory mask is particularly easy to put on or remove and, on account of the locking system, ensures a secure fit of the mask body. In addition, the indirect actuation of the locking element via the actuation element permits a particularly ergonomic embodiment of the actuation element independently of the structural characteristics of the locking element. This can significantly improve the operating comfort compared to an embodiment without such an actuation element.

In a third aspect the invention provides a respiratory mask for supplying a patient with respiratory gas. The respiratory mask comprises: a mask body for applying to the mouth and/or nose of the patient, wherein the mask body comprises a first coupling receptacle, a second coupling receptacle, and a tube connector for the attachment of a breathing tube; a head harness for fixing the mask body with respect to the patient's face; a first coupling element for coupling the head harness to the first coupling receptacle; a second coupling element for coupling the head harness to the second coupling receptacle. Each coupling element comprises a coupling portion for coupling the coupling element to the respective coupling receptacle, and a strap receptacle for fastening a strap of the head harness. The coupling portion of at least one (or each) of the two coupling elements comprises a hook-shaped hook portion which can be hooked onto the respective coupling receptacle to form a hooking arrangement for fastening the head harness to the mask body. Furthermore, the coupling portion comprises a wing-shaped actuation portion which is firmly connected to the hook portion and which is arranged such that, when the mask body is fixed with respect to the patient's face by means of the head harness (and the hook portion is hooked onto the respective coupling receptacle), the actuation portion protrudes from the rest of the coupling portion in such a way that said actuation portion can be pivoted with at least one finger in a pivoting direction away from the respective coupling receptacle. The hooking arrangement is configured in such a way that, when the mask body is fixed with respect to the patient's face by means of the head harness, the hook portion is pressed against the respective coupling receptacle by a tensile force exerted by the head harness, in order to prevent unintentional release of the hooking arrangement, and can be moved out of the respective coupling receptacle by pivoting of the actuation portion in the pivoting direction, in order to enable a controlled release of the hooking arrangement.

A hooking arrangement of this kind is a viable alternative to the plug connection described above.

It will be noted that features of the respiratory mask according to the first aspect of the invention can also be features of the respiratory mask according to the second or third aspect of the invention (and vice versa).

A “respiratory mask” can be understood, for example, as an oral mask, a nasal mask, or a combination of an oral and a nasal mask, also called a full-face mask. The respiratory mask may be suitable for use with a ventilator. A “ventilator” can be understood as a device for invasive and/or non-invasive ventilation of the patient and/or for assisting the patient in coughing. The ventilator can also be an anesthesia machine.

“Sagittal axis” can be understood as an anteroposterior axis. “Longitudinal axis” can be understood as a craniocaudal axis (orthogonal to the anteroposterior axis). If the patient is standing upright, the sagittal axis can coincide with a horizontal and the longitudinal axis with a vertical.

A “strap” can generally be understood as an elongate portion of the head harness for exerting a suitable tensile force on the coupling elements. Such a strap can be configured to be particularly elastic. For example, the strap can be configured as a rubber and/or textile strap. By appropriately adjusting the strap or straps, it is possible to change the position and/or orientation of each coupling element, in particular of the strap receptacle(s) and of the coupling portion, relative to the patient's head. The coupling of the mask body to the coupling elements can be such that an adjustment of the strap or straps in a corresponding manner affects the position and/or orientation of the mask body relative to the patient's face. For this purpose, the coupling elements can for example be configured to be torsionally rigid and/or can be rigidly coupled to the mask body.

The first strap and the second strap can be single straps or form a strap assembly. The straps of the strap assembly can be connected directly and/or indirectly to one another (for example, via one or more additional portions of the head harness). Advantageously, the first strap and the second strap can be configured to be adjustable independently of each other in their length and/or tensile force.

The head harness and/or the first strap and the second strap can additionally be reinforced at least in some places with a material that is particularly stiff in comparison to rubber or textile, for example a thermoplastic or thermosetting plastic. The head harness can also comprise more than two straps.

It is possible that the end portion of each strap can be passed through the respective strap receptacle and connected to the remaining portion of the strap and/or to the remaining portion of the head harness to form a loop. The connection can, for example, be a touch-and-close connection and/or can be realized using clasps, buttons, hooks, eyelets, belts or knots.

The mask body can be configured such that it encloses the mouth and/or the nose (or at least the nostrils) of the patient in an airtight manner when it is fixed with respect to the patient's face by means of the head harness. In this case, the mask body, together with the enclosed part of the face, can define a respiratory gas space which can be filled with or emptied of respiratory gas via the tube connector and the breathing tube attached to the latter. During use of the respiratory mask, the mask body can be applied indirectly to the patient's face (for example via a mask bead and/or a mask cushion) and/or can be applied directly to the patient's face.

The coupling receptacles can be arranged on both sides of the mask body. For example, the mask body can have a coupling receptacle on a left-hand side and a further coupling receptacle on a right-hand side (seen from the perspective of the patient wearing the mask).

The coupling elements, in combination with the head harness, have the effect that a contact pressure with which the mask body is pressed against the face, and/or an inclination of the mask body with respect to the sagittal axis and/or the longitudinal axis and/or a transverse axis orthogonal to the sagittal axis and to the longitudinal axis of the patient's body, can be adapted to the individual facial contours of the patient. In this way it is possible to avoid unpleasant or even painful pressure points. For this purpose, each coupling element (or at least a part thereof) can be configured as a lever for additionally applying a certain torque to the mask body. This torque, more precisely its magnitude and direction, may depend on the tensile forces applied to the strap receptacles by the head harness, more precisely on their respective magnitude and direction. The head harness is therefore not connected to the mask body directly, but indirectly via the coupling elements. For example, during use of the respiratory mask, the first coupling element can be directed to the left-hand side of the face and the second coupling element to the right-hand side of the face.

Each coupling element can, for example, be configured such that a first distance between the first strap receptacle and the second strap receptacle (more precisely between a first force application point, where a first tensile force is applied by the first strap fastened to the first strap receptacle, and a second force application point, where a second tensile force is applied by the second strap fastened to the second strap receptacle) and a second distance between the first strap receptacle and the coupling portion (more precisely between the first force application point and a third force application point where a reaction force is applied by the coupling receptacle of the mask body coupled to the coupling portion) correspond to each other or deviate by not more than 20 percent, preferably not more than about 10 percent, from each other. Alternatively, the first distance can also be considerably greater (for example by more than about 20 percent or at least about 50 percent) than the second distance (or vice versa). In this context, “first distance” can be understood to mean a length of a first lever arm. Similarly, “second distance” can be understood to mean a length of a second lever arm. For example, an angle enclosed by the two lever arms can be between about 60 and about 120 degrees, between about 80 and about 100 degrees, or between about 45 and about 90 degrees, in particular about 70 degrees.

The coupling elements can advantageously be made from a material that is particularly stiff compared to rubber or textile, for example a thermoplastic or thermosetting plastic, a metal, or a combination of at least two of these examples.

The coupling elements can be of the same design. Alternatively, the coupling elements can be differently shaped, in particular in such a way that they can only be coupled to exactly one of the coupling receptacles, which can further simplify the application of the respiratory mask. Each coupling element can also comprise more than two strap receptacles and/or more than one coupling portion.

The mask body and the two coupling elements can also be in one piece and/or made from the same material, for example a thermoplastic material.

The patient's head can also include the back of the neck.

Various embodiments of the invention are described below. These embodiments should not be understood as restricting the scope of the invention.

According to one embodiment, each coupling element can be configured such that, when the mask body is fixed with respect to the patient's face by means of the head harness (i.e. during use of the respiratory mask), the first strap receptacle is arranged at least partially opposite a part of the lower jaw of the patient, in particular opposite a ramus of the lower jaw, and/or outside the patient's cheek region. This makes it possible to tension the first strap below the ears across the back of the neck and/or the occipital bone of the patient. Thus, the first strap receptacle or the first strap can be positioned at least for the most part outside the patient's field of vision or line of sight.

According to one embodiment, each coupling element can be configured such that, when the mask body is fixed with respect to the patient's face by means of the head harness, the coupling portion coupled to the respective coupling receptacle, and viewed in the direction of the longitudinal axis, is arranged at least partially between the first strap receptacle and the second strap receptacle. In addition or alternatively, the second strap receptacle, viewed in the direction of the sagittal axis, can be arranged at least partially between the first strap receptacle and the coupling portion coupled to the respective coupling receptacle.

It is possible that, when viewed in the direction of the longitudinal axis, the first strap receptacle is arranged at least partially below the coupling portion coupled to the respective coupling receptacle, and/or the second strap receptacle is arranged at least partially above the coupling portion coupled to the respective coupling receptacle.

In addition, the second strap receptacle, viewed in the direction of the longitudinal axis, can be arranged at least partially above the mask body and/or above the patient's ears and/or above the patient's eyes and/or, viewed in the direction of the sagittal axis, at least partially between the eye and the ear on the respective side of the patient's face.

This makes it possible to tension the second strap above the ears and/or across the back of the head, for example across the parietal bone. Thus, the second strap receptacle or the second strap can be positioned at least for the most part outside the patient's field of view or line of sight.

In addition, viewed in the direction of the sagittal axis, the second strap receptacle can be arranged (discernibly) closer to the mask body than the first strap receptacle (or vice versa). For example, a distance of the first strap receptacle to the mask body in the direction of the sagittal axis can deviate by at least about 10 percent, at least about 20 percent or at least about 40 percent from a corresponding distance of the second strap receptacle.

It is also possible that the first strap receptacle and the second strap receptacle at least partially overlap each other, viewed in the direction of the longitudinal axis.

According to one embodiment, when the mask body is fixed with respect to the patient's face by means of the head harness, the first strap exerts a first tensile force on the first strap receptacle of each coupling element and the second strap exerts a second tensile force on the second strap receptacle of each coupling element. In this case, it is possible that:
a component of the first tensile force acting in the direction of the sagittal axis is (considerably) greater than, preferably at least about twice as great as, a component of the first tensile force acting in the direction of the longitudinal axis, and/or
a component of the second tensile force acting in the direction of the sagittal axis is (considerably) greater than, preferably at least about twice as great as, a component of the second tensile force acting in the direction of the longitudinal axis, and/or
the first tensile force and the second tensile force are parallel to each other within a predefined tolerance range, and/or
the first tensile force is directed to the back of the neck and/or the occipital bone of the patient, and/or
the second tensile force is directed to a head region away from the back of the neck and/or occipital bone of the patient, in particular to the patient's parietal bone.

For example, the sagittal component of the first or second tensile force can be at least about 120%, at least about 200% or even at least about 300% of the respective longitudinal component. In some cases, it is possible that the longitudinal component of the first and/or second tensile force is equal to zero or negligible in comparison to the respective sagittal component. In other words, the first and/or second tensile force can in some cases act exclusively in the direction of the sagittal axis.

The predefined tolerance range can, for example, correspond to a permissible angular deviation of no more than plus/minus about 10 degrees, preferably no more than plus/minus about 5 degrees, between the action lines of the two tensile forces.

According to one embodiment, each coupling element can comprise a first arm and a second arm. The first arm and the second arm can be connected to each other in a transition portion in such a way that they form an L shape or V shape. In this case, the first strap receptacle can be arranged at least partially in the transition portion, and/or the second strap receptacle can be arranged at an end of the second arm facing away from the transition portion, and/or the coupling portion can be arranged at an end of the first arm facing away from the transition portion. This permits a particularly compact, yet sufficiently stable design of the coupling elements. Alternatively, the first arm and the second arm can also be connected to each other to form a T shape.

According to one embodiment, each first strap receptacle and/or each second strap receptacle can comprise a receiving opening formed in the material of the respective coupling element, for insertion of the respective strap. The receiving opening can be at least partially limited by an edge portion which is curved (for example in the shape of an arc of a circle) in a circumferential direction of the receiving opening. The strap fastened to the respective strap receptacle can at least partially wrap around the edge portion. For example, the strap can form a loop around the edge portion when the mask body is fixed with respect to the patient's face by means of the head harness. An orientation of a longitudinal axis of the respective strap relative to the sagittal axis can be varied by the part of the respective strap surrounding the edge portion being moved along the edge portion. In this way, an angle enclosed by the sagittal axis and the longitudinal axis of the strap can be adapted very easily and conveniently to the individual contours of the patient's face or head.

According to one embodiment, the coupling portion of at least one (or each) of the two coupling elements can be connectable to the respective coupling receptacle to form a (releasable) plug connection. In this case, the respiratory mask can further comprise a locking element movably mounted on the mask body (for example on the coupling receptacle) and/or on the respective coupling element (for example on the coupling portion) between a locking position and an unlocking position. The locking element can be configured to lock the plug connection in the locking position, so that a movement of the coupling portion in a separation direction away from the respective coupling receptacle is not possible, and to unlock it in the unlocking position, so that a movement of the coupling portion in the separation direction is possible (at least to a limited extent).

In other words, the coupling portion can be latched onto the respective coupling receptacle by means of a latching mechanism. The latching mechanism can be configured to latch the coupling portion in a predetermined position, or in one of several possible predetermined positions, to the coupling receptacle by means of spring force. Thus, the coupling elements can be coupled to the mask body in a very simple and convenient way and released from it again.

According to one embodiment, the respiratory mask can further comprise an actuation element movably mounted on the mask body and/or on the respective coupling element between a rest position and an actuation position. The actuation element may be suitable for actuating the locking element when the plug connection is locked. The actuation element can be configured to press against the locking element when moved to the actuation position, such that the locking element is moved to the unlocking position. Such indirect actuation of the locking element permits a particularly ergonomic embodiment of the actuation element independently of the structural characteristics of the locking element. This can further improve the operating comfort.

According to one embodiment, the actuation element can be configured to limit a movement path of the coupling portion during movement in the separation direction in the actuation position, such that the coupling portion can be moved only to an intermediate position relative to the respective coupling receptacle, and not to limit it in the rest position, such that the coupling portion can be moved beyond the intermediate position. The plug connection can be disconnected by moving the coupling portion beyond the intermediate position. In other words, the coupling portion and the coupling receptacle of the plug connection can be (completely) separated from each other only when the coupling portion is moved beyond the intermediate position. This has the effect that the plug connection is not immediately disconnected when the actuation element is first actuated (for example pressed). Under certain circumstances, the coupling portion could be thrown off from the coupling receptacle in an uncontrolled manner on account of the tensile forces exerted by the head harness. This can be avoided if the plug connection is (completely) disconnected only when the actuation element is moved back to the rest position after having moved to the actuation position. This allows controlled separation of the plug connection.

According to one embodiment, the locking element, by movement of the coupling portion to the intermediate position, can be positioned opposite a stop edge of the mask body (for example of the coupling receptacle) and/or of the respective coupling element (for example of the coupling portion). The stop edge can be configured such that the locking element positioned opposite the stop edge is prevented from returning to the locking position. For this purpose, the locking element positioned opposite the stop edge can bear in a suitable manner against the stop edge, i.e. touch the latter (directly). The movement of the coupling portion to the intermediate position, when the plug connection is unlocked, can be effected, for example, solely on the basis of the tensile forces exerted by the head harness. Due to the fact that the locking element is mechanically blocked in a suitable manner in the intermediate position, unintentional re-locking of the plug connection when moving the actuation element back to the rest position can be prevented.

According to one embodiment, the actuation element, in the actuation position, can engage in a preferably wedge-shaped depression in the mask body (for example in the coupling receptacle) and/or in the respective coupling element (for example in the coupling portion). The actuation element can be movable out of the depression by moving back to the rest position. The depression can be bounded, in a direction transverse to the separation direction, by two obliquely oriented boundary edges. The two boundary edges can be oriented obliquely with respect to each other such that they run toward each other when viewed in a direction counter to the separation direction. The actuation element can be configured in such a way that a portion of the actuation element engaging in the depression is moved toward the boundary edges upon movement of the coupling portion in the separation direction and, when the coupling portion reaches the intermediate position, abuts on both sides at the boundary edges, such that further movement of the coupling portion beyond the intermediate position is not possible.

According to one embodiment, the actuation element can be configured as an actuation tab. The actuation tab can be connected at one of its ends to the mask body (for example to the coupling receptacle) and/or to the respective coupling element (for example to the coupling portion). The actuation tab, by elastic bending of its other end, for example the unconnected end, is movable into the actuation position, such that a spring force acts on the actuation tab in the actuation position to return the actuation tab to the rest position. This has the effect that, when released, the actuation element moves back automatically to the rest position, unless otherwise prevented from doing so.

According to one embodiment, the locking element can be configured as a locking tab. The locking tab can be connected at one of its ends to the mask body (for example to the coupling receptacle) and/or to the respective coupling element (for example to the coupling portion). The locking tab, by elastic bending of its other end, for example the unconnected end, is movable into the unlocking position, such that a spring force acts on the locking tab in the unlocking position to return the locking tab to the locking position. This has the effect that, when released, the locking element moves back automatically to the locking position, unless otherwise prevented from doing so.

A “tab”, as in “locking tab” or “actuation tab”, can be understood to mean, for example, a plate-shaped or finger-shaped element.

According to one embodiment, the coupling portion of at least one (or each) of the two coupling elements can comprise a hook-shaped hook portion which can be hooked onto the respective coupling receptacle to form a hooking arrangement for fastening the head harness to the mask body. Furthermore, the coupling portion can comprise a wing-shaped actuation portion which is firmly connected to the hook portion and which is arranged such that, when the mask body is fixed with respect to the patient's face by means of the head harness (and the hook portion is hooked onto the respective coupling receptacle), the actuation portion protrudes from the rest of the coupling portion in such a way that said actuation portion can be pivoted with at least one finger in a pivoting direction away from the respective coupling receptacle. The hooking arrangement can be configured in such a way that, when the mask body is fixed with respect to the patient's face by means of the head harness, the hook portion is pressed against the respective coupling receptacle by a tensile force exerted by the head harness, in order to prevent unintentional release of the hooking arrangement, and can be moved out of the respective coupling receptacle by pivoting of the actuation portion in the pivoting direction, in order to enable a controlled release of the hooking arrangement.

According to one embodiment, the respective coupling element, viewed in the longitudinal direction thereof, can be divided into at least a first longitudinal portion, a second longitudinal portion and a third longitudinal portion. The second longitudinal portion can be arranged between the first longitudinal portion and the third longitudinal portion and/or can transition directly into the first longitudinal portion and/or directly into the third longitudinal portion. With respect to a longitudinal axis of the respective coupling element, the first longitudinal portion can protrude transversely, obliquely or in a straight line from one end (the end facing away from the third longitudinal portion) of the second longitudinal portion and/or can comprise the actuation portion. For example, the first longitudinal portion can be configured as the actuation portion. By contrast, the second longitudinal portion can comprise the hook portion. For example, the second longitudinal portion can be configured as the hook portion. The third longitudinal portion can be a remaining longitudinal portion of the respective coupling element. It is possible that the second longitudinal portion is connected to the third longitudinal portion via a fourth longitudinal portion, which can comprise a bending portion described in more detail below or can be configured as a bending portion described in more detail below.

According to one embodiment, the coupling portion of the respective coupling element can further comprise a nose, which is configured to engage in a recess, complementary to the nose, of the respective coupling receptacle and/or of the mask body when the hook portion is hooked onto the respective coupling receptacle. The nose can have a special stop surface and can further be configured such that the stop surface of the nose engaging in the recess, upon movement of the coupling portion relative to the respective coupling receptacle in at least one direction of movement, preferably parallel to an effective direction of the tensile force exerted by the head harness, strikes against a special counter-surface of the recess, such that further movement of the coupling portion relative to the respective coupling receptacle in the respective direction of movement is not possible. In this case, a movement of the coupling portion relative to the respective coupling receptacle in at least one further direction of movement deviating from the direction of movement, preferably orthogonal or oblique to the direction of movement, is still possible to a limited extent without the hooking arrangement being released. On account of such limited play in the respective further direction of movement, it is possible, for example, to prevent unintentional release of the hooking arrangement when using the respiratory mask on faces with different widths. For example, the nose can protrude in a direction toward the hook portion from another part of the coupling portion or of the respective coupling element. In addition or alternatively, the hook portion can at least partially overhang the nose. It is possible that the nose and the hook portion protrude from the same side of each coupling element. For example, the nose can be arranged in the aforementioned second longitudinal portion together with the hook portion.

According to one embodiment, the nose can be at least partially wedge-shaped, in order to allow the nose to slide out of the recess, when the hooking arrangement is released in a controlled manner, solely due to the tensile force exerted by the head harness. This can further improve the operating comfort.

According to one embodiment, the hook portion can have a rounded hook inner contour, which can engage around a rounded coupling (outer) contour of the respective coupling receptacle, complementary to the hook inner contour, when the hook portion is hooked onto the respective coupling receptacle. In this case, the hooking arrangement can be configured in such a way that, when the mask body is fixed with respect to the patient's face by means of the head harness, the hook inner contour engaging around the coupling contour is pressed against the coupling contour by the tensile force exerted by the head harness, in order to prevent the unintentional release of the hooking arrangement. It is possible that, during the pivoting of the actuation portion in the pivoting direction, the hook inner contour engaging around the coupling contour is moved at least partially relative to the coupling contour, for example at least partially along the coupling contour, in order to enable a pivoting movement of the hook portion out of the respective coupling receptacle. Such a guided pivoting movement can further simplify the controlled release of the hooking arrangement.

According to one embodiment, a free end of the hook portion can additionally be latched onto and/or in the respective coupling receptacle to form the hooking arrangement, so that a defined minimum force is required for controlled release of the hooking arrangement or for pivoting the actuation portion in the pivoting direction. The minimum force for releasing the latching, for example by correspondingly pressing the actuation portion in the pivoting direction, can be considerably greater than a minimum force for merely pivoting the actuation portion in the pivoting direction without such additional latching. For example, when forming the hooking arrangement, the free end, for instance in the form of a tip of the hook portion, can additionally latch with form-fit and/or force-fit engagement in a correspondingly shaped depression in the respective coupling receptacle, in order to latch the free end to the respective coupling receptacle.

According to one embodiment, the coupling portion of at least one (or each) of the two coupling elements can be rigidly connected to the respective coupling receptacle in the coupled state (i.e. when the coupling portion is coupled to the respective coupling receptacle). In other words, in the coupled state, no (significant) movement of the respective coupling element relative to the mask body is possible. In contrast to an articulated coupling, this has the effect that the mask body can be subjected to a certain torque via the respective coupling element. Thus, for example, by applying a corresponding torque even in the case of a larger mask body of a full-face mask, an even distribution of the contact pressure over the nose and mouth region can be ensured, without this requiring a potentially annoying forehead support or additional straps. For example, the hooking arrangement can be configured with a suitable side guide in order to prevent the mask body from unintentionally twisting relative to the coupling portion when wearing the respiratory mask. It is thereby possible, for example, to prevent the mask body from tilting away from the face, for example due to a sharp movement of the head.

According to one embodiment, the coupling portion of at least one (or each) of the two coupling elements can comprise a wedge-shaped outer contour for insertion into the respective coupling receptacle. In this case, the respective coupling receptacle can comprise a funnel-shaped inner contour, complementary to the outer contour, for receiving the outer contour. This permits self-centering of the coupling portion with respect to the coupling receptacle during insertion. The operating comfort can thus be further improved.

According to one embodiment, the coupling portion of at least one (or each) of the two coupling elements can be connected to a remaining longitudinal portion of the respective coupling element via a bending portion. The bending portion can be particularly flexible in comparison to the coupling portion and to the remaining longitudinal portion, in order to allow the coupling portion to be angled relative to the remaining longitudinal portion. This makes it possible to use the respiratory mask, in particular a variant of the respiratory mask with a hooking arrangement as described above and below, on faces with (clearly) different widths, without needing to specially adapt the respiratory mask to the respective face width. As mentioned above, the bending portion can, for example, be a fourth longitudinal portion of the respective coupling element, which fourth longitudinal portion is arranged between the second longitudinal portion and the third longitudinal portion.

According to one embodiment, a thickness of the bending portion, viewed in the longitudinal direction of the respective coupling element, can vary between at least two different thickness values, according to a predetermined (preferably periodic or sinusoidal) thickness profile. For example, the thickness of the bending portion can vary in an undulating shape when viewed in the longitudinal direction of the respective coupling element. In other words, the bending portion can be configured, for example, as a thin point in comparison to the coupling portion and to the remaining longitudinal portion. In addition or alternatively, the bending portion can be made at least partially from a different material than the coupling portion and/or the remaining longitudinal portion and/or at least partially from a particularly soft material such as silicone or a (preferably thermoplastic) elastomer. Alternatively, the bending portion can be configured as a mechanical joint, i.e. as a joint having at least two interconnected joint parts movable in at least one direction (for example in the form of a hinge or a ball joint). This permits a particularly flexible and yet stable mechanical connection between the coupling portion and the remaining is longitudinal portion. In addition, such a bending portion can be produced particularly easily in large numbers, for example—depending on the design—together with the rest of the coupling element in an injection molding process.

According to one embodiment, the coupling portion of at least one (or each) of the two coupling elements, preferably the actuation portion and/or the hook portion, can comprise a release line receptacle for the attachment of a release line to the coupling portion. With the aid of the release line attached to the release line receptacle, the hooking arrangement can be quickly and safely released in an emergency by suitably pulling on the release line, for example in a pulling direction that runs parallel to the effective direction of the tensile force exerted by the head harness, without for this purpose having to bring a hand to the actuation portion.

### BRIEF DESCRIPTION OF THE DRAWINGS

Embodiments of the invention are described below with reference to the accompanying figures. Neither the description nor the figures should be understood as limiting the scope of the invention. In the drawings:

FIG. 1 shows a perspective view of a respiratory mask according to an embodiment of the invention.

FIG. 2 shows a side view of a respiratory mask according to an embodiment of the invention.

FIG. 3 shows a side view of the head of a patient wearing the respiratory mask of FIG. 2.

FIG. 4 shows an enlarged view of a coupling element of a respiratory mask according to an embodiment of the invention.

FIG. 5 shows a detailed view of a coupling receptacle and of a coupling portion of a respiratory mask according to an embodiment of the invention in the uncoupled state.

FIG. 6 shows a sectional view of the coupling receptacle and of the coupling portion of FIG. 5 in the coupled state.

FIG. 7 shows the respiratory mask of FIG. 2 from the front.

FIG. 8 shows the respiratory mask of FIG. 2 from behind.

FIG. 9 shows the respiratory mask of FIG. 2 from above.

FIG. 10 shows the respiratory mask of FIG. 2 from below.

FIG. 11 shows a mask body and coupling elements of a respiratory mask according to an embodiment of the invention, in which the coupling elements are hooked into the mask body.

FIG. 12 shows a plan view of the mask body and the coupling elements from FIG. 11.

FIG. 13 shows a cross section through the mask body and the coupling elements from FIG. 11 and FIG. 12.

FIG. 14 shows an enlarged view of a region labeled “A” in FIG. 13.

FIG. 15 shows a perspective view of one of the coupling elements of FIG. 11 to FIG. 13.

FIG. 16 shows a perspective view of the mask body from FIG. 11 to FIG. 13.

The figures are purely schematic and not true to scale. If identical reference signs are used in different drawings, then these reference signs designate identical or identically acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

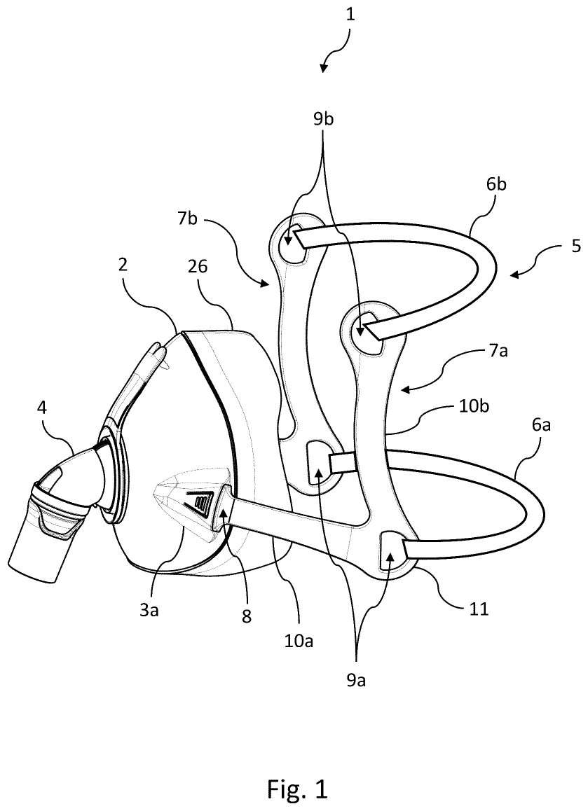

FIG. 1 shows a perspective view of a respiratory mask 1 for supplying a patient with respiratory gas from a respiratory gas source.

The respiratory mask 1 comprises a mask body 2 having a first coupling receptacle 3a, a second coupling receptacle 3b, and a tube connector 4 for the attachment of a breathing tube connected to the respiratory gas source. In this example, the respiratory mask 1 is a full-face mask. Accordingly, the mask body 2 encloses the mouth and nose of the patient during use of the respiratory mask 1. The mask body 2 can be correspondingly curved for this purpose, so that it forms a hollow body in which the mouth and nose can be placed.

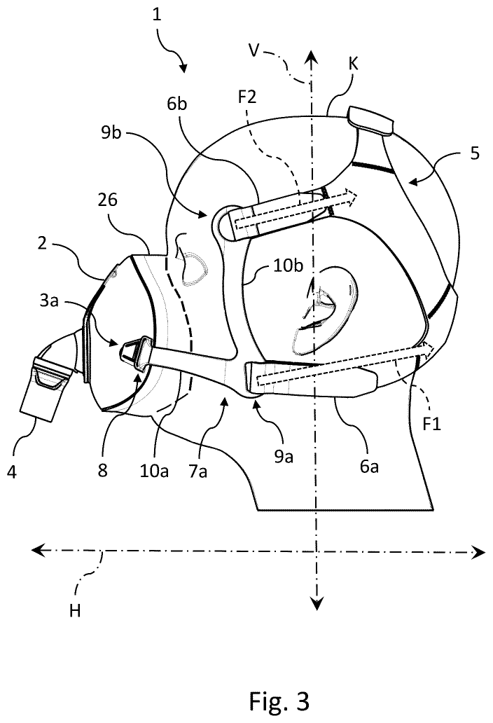

Furthermore, the respiratory mask 1 comprises a head harness 5 for fixing the mask body 2 with respect to the patient's face. In this example, the head harness 5 comprises a first strap 6a and a second strap 6b for tensioning around the patients' head K (see also FIG. 3).

In addition, the respiratory mask 1 comprises a first coupling element 7a for coupling the head harness 5 to the first coupling receptacle 3a, and a second coupling element 7b for coupling the head harness 5 to the second coupling receptacle 3b.

Each coupling element 7a, 7b comprises a coupling portion 8 for coupling the coupling element 7a, 7b to the respective coupling receptacle 3a, 3b, a first strap receptacle 9a for fastening the first strap 6a, and a second strap receptacle 9b for fastening the second strap 6b. For example, in the coupled state, the coupling portion 8 can be rigidly connected to the respective coupling receptacle 3a, 3b.

As can be seen from FIG. 3, each coupling element 7a, 7b is configured such that, when the mask body 2 is fixed with respect to the patient's face by means of the head harness 5, the first strap receptacle 9a, viewed in the direction of a sagittal axis (H) of the patient (here viewed in the direction of a horizontal H), is arranged at least partially between the chin and an ear of the patient and, viewed in the direction of a longitudinal axis V of the patient orthogonal to the sagittal axis (H) (here viewed in the direction of a vertical V), is arranged at least partially between the chin and the temporal region of the patient. In this case, the second strap receptacle 9b is arranged at least partially opposite the temporal region of the patient.

In this example, the first strap receptacle 9a is arranged at least partially opposite a part of the patient's lower jaw, in particular opposite a ramus of the lower jaw. In addition, the coupling portion 8 coupled to the respective coupling receptacle 3a, 3b can be arranged, when viewed in the direction of the longitudinal axis V, at least partially between the first strap receptacle 9a and the second strap receptacle 9b. In this case, the second strap receptacle 9b, viewed in the direction of the sagittal axis H, can be arranged at least partially between the coupling portion 8 and the first strap receptacle 9a. In other words, viewed in the direction of the sagittal axis H, the second strap receptacle 9b can be arranged discernibly closer to the mask body 2 than the first strap receptacle 9a.

During use of the respiratory mask 1, the first strap 6a exerts a first tensile force F1 on the first strap receptacle 9a of each coupling element 7a, 7b. In a corresponding manner, the second strap 6b exerts a second tensile force F2 on the second strap receptacle 9b of each coupling element 7a, 7b.

In this case, the first tensile force F1 can be directed to the back of the neck and/or to the occipital bone of the patient, whereas the second tensile force F2 can be directed to a head region away from the back of the neck and/or from the occipital bone of the patient, in particular directed to the parietal bone of the patient. Accordingly, the first strap 6a below the ears can be tensioned around the back of the neck and/or the occipital bone, and/or the second strap 6b above the ears can be tensioned around the head region located away from the back of the neck and/or from the occipital bone, in particular around the parietal bone.

It is possible that a component of the first tensile force F1 acting in the direction of the sagittal axis H is considerably greater than, preferably at least twice as great as, a component of the first tensile force F1 acting in the direction of the longitudinal axis V. This can also be the case for the second tensile force F2.

In addition, as indicated here, the first tensile force F1 and the second tensile force F2 can be oriented (approximately) parallel to each other.

On account of the two tensile forces F1, F2, the mask body 2 is pressed against the face in such a way that an airtight connection is established between the mask body 2 and the face.

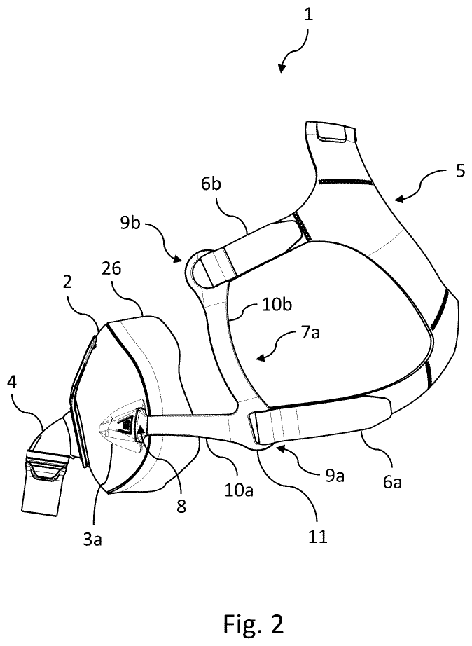

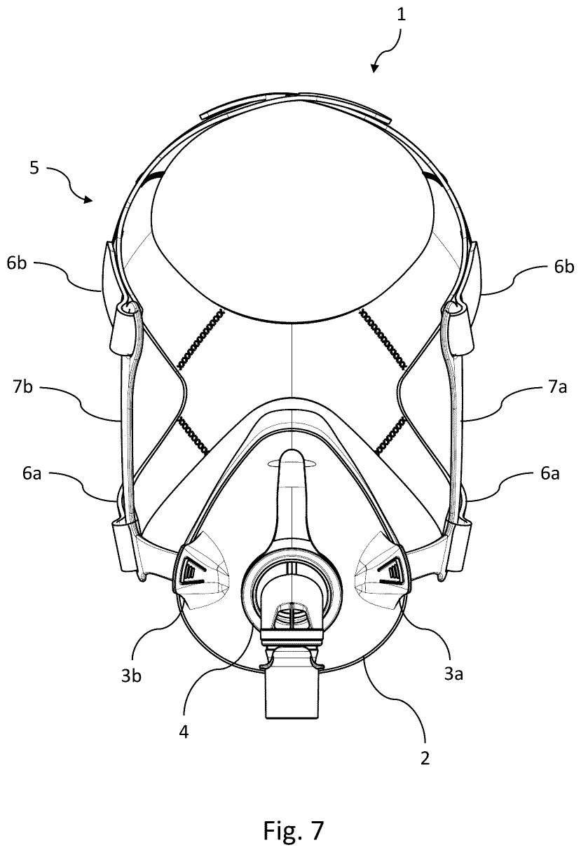

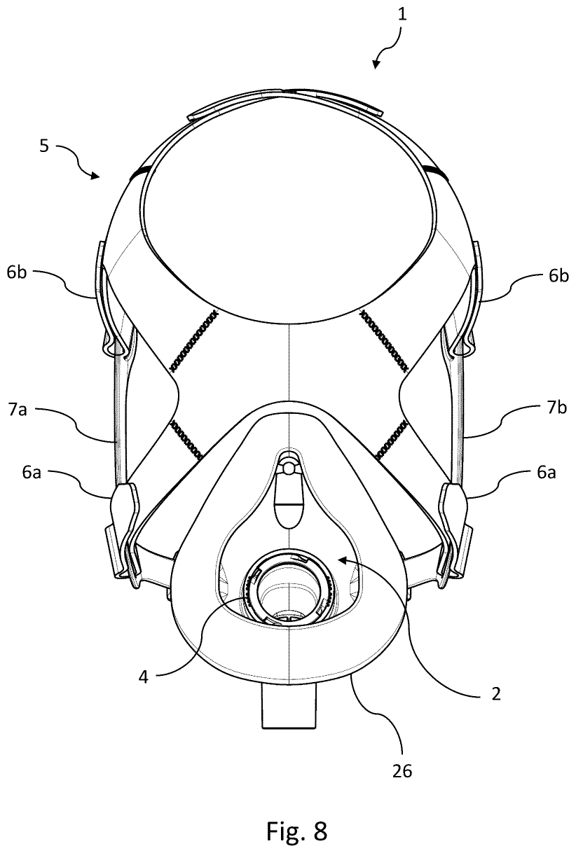

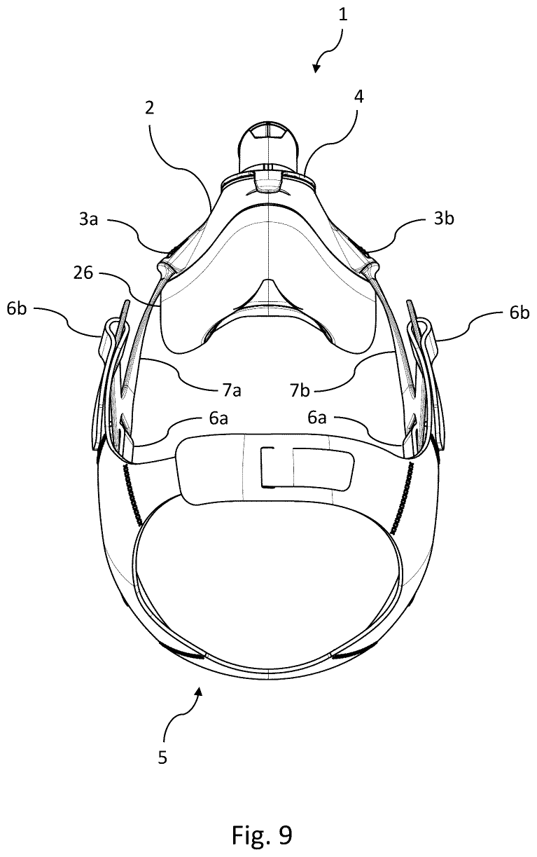

As is shown in FIG. 1, the first strap 6a and the second strap 6b can be straps separate from each other or, as is shown in FIG. 2, FIG. 3 and FIG. 7 to FIG. 10, can be combined with each other to form a strap assembly.

Optionally, each coupling element 7a, 7b can comprise a first arm 10a and a second arm 10b, which can be connected to each other in an L shape or V shape and can transition into each other in a transition portion 11. As is shown here, the first strap receptacle 9a can be arranged at least partially in the transition portion 11, the second strap receptacle 9b can be arranged at an end of the second arm 10b facing away from the transition portion 11, and the coupling portion 8 can be arranged at an end of the first arm 10a facing away from the transition portion 11.

In addition, the two strap receptacles 9a, 9b of each coupling element 7a, 7b can comprise a receiving opening 12 formed in the material of the respective coupling element 7a, 7b, for insertion of the respective strap 6a, 6b. For example, the end portions of the straps 6a, 6b can be passed through the corresponding receiving opening 12 and suitably fastened to a remaining portion of the head harness 5, for example a remaining portion of the respective strap 6a, 6b, to form a loop. The fastening can preferably be effected via a touch-and-close connection.

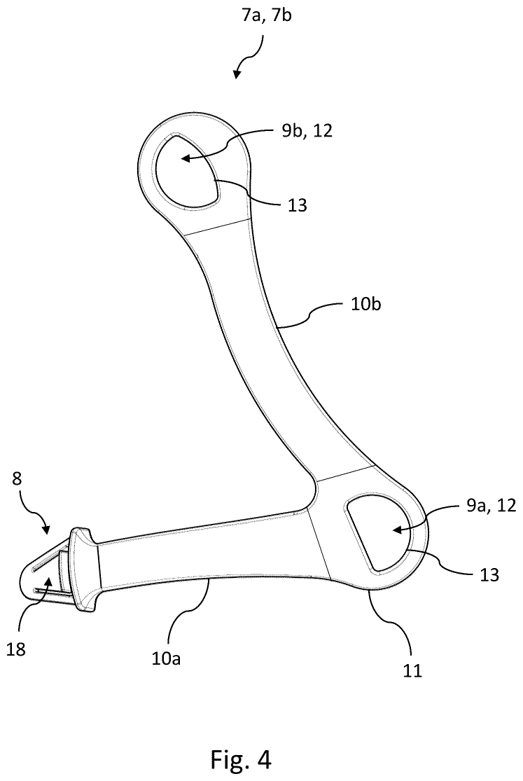

As can be seen in FIG. 4, the receiving opening 12 can be at least partially limited by an edge portion 13 which is curved, for example in the shape of an arc of a circle, in a circumferential direction of the receiving opening 12. The strap 6a, 6b fastened to the respective strap receptacle 9a, 9b, for example connected to form a loop, can at least partially wrap around the edge portion 13. Accordingly, an orientation of a longitudinal axis of the respective strap 6a, 6b relative to the sagittal axis H can be varied by the part of the respective strap 6a, 6b surrounding the edge portion 13 being moved along the edge portion 13. The edge portion 13 can be considerably longer than the part of the respective strap 6a, 6b surrounding the edge portion 13 is wide. This allows sufficient adjustment in both directions.

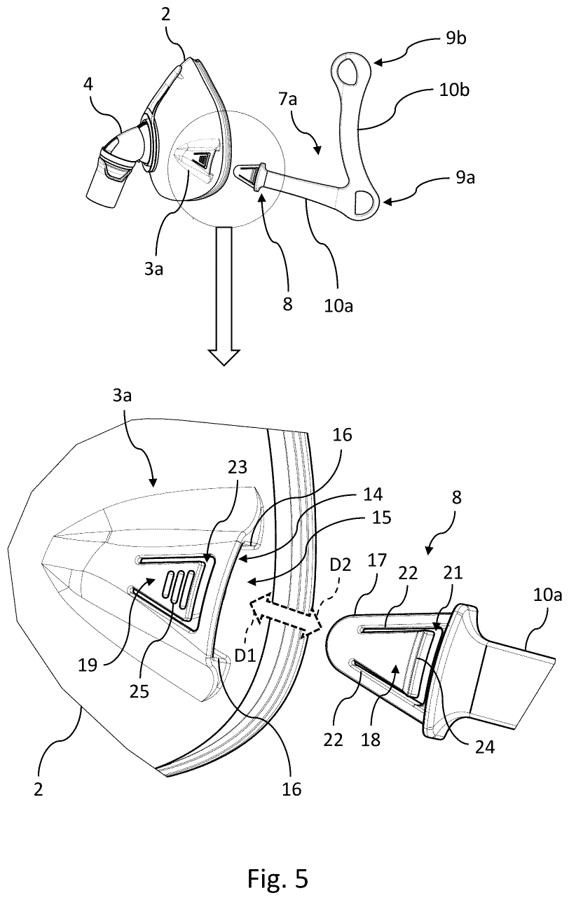

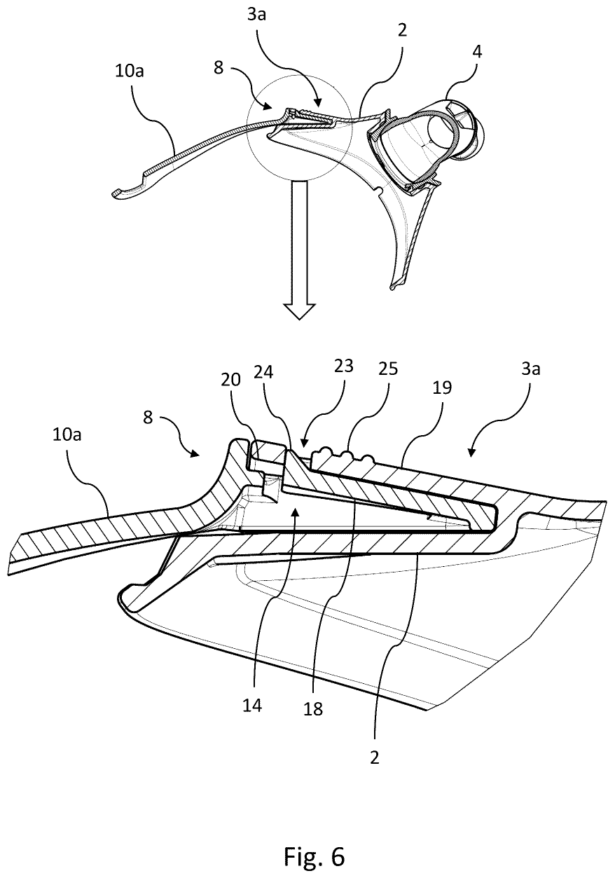

As is shown in FIG. 5 and FIG. 6, using the example of the first coupling receptacle 3a, each coupling receptacle 3a, 3b can comprise a receiving space 14 with an insertion opening 15 for insertion of the respective coupling portion 8 in an insertion direction D1.

The receiving space 14 can, for example, have a funnel-shaped inner contour 16. Accordingly, the coupling portion 8 can be formed with a wedge-shaped outer contour 17, complementary to the inner contour 16, for insertion into the inner contour 16. This permits self-centering of the coupling portion 8 relative to the coupling receptacle 3a, 3b upon insertion through the insertion opening 15.

In addition, the coupling portion 8 can be connectable to the coupling receptacle 3a, 3b to form a plug connection.

For this purpose, a locking element 18 can be movably mounted on the coupling portion 8 between a locking position and an unlocking position. The locking element 18 can be configured to lock the plug connection in the locking position, so that a movement of the coupling portion 8 in a separation direction D2 (counter to the insertion direction D1) away from the respective coupling receptacle 3a, 3b is not possible, and to unlock it in the unlocking position, so that it is possible to move the coupling portion 8 in the separation direction D2 (at least to a limited extent).

Furthermore, an actuation element 19 can be movably mounted on the coupling receptacle 3a, 3b between a rest position and an actuation position. The actuation element 19 serves to actuate the locking element 18 when the plug connection is locked. The actuation element 19 can be configured to press against the locking element 18 when moving into the actuation position, such that the locking element 18 is moved into the unlocking position.

In addition, the actuation element 19 can be configured to limit a movement path of the coupling portion 8 during movement in the separation direction D2 in the actuation position, such that the coupling portion 8 can be moved only to an intermediate position relative to the respective coupling receptacle 3a, 3b, and not to limit it in the rest position, such that the coupling portion 8 can be moved beyond the intermediate position. The plug connection is not completely disconnected until the coupling portion 8 is moved beyond the intermediate position.

It is possible that the locking element 18, by movement of the coupling portion 8 to the intermediate position, can be positioned opposite a stop edge 20 of the coupling receptacle 3a, 3b. The stop edge 20 can be configured such that the locking element 18 positioned opposite the stop edge 20, for example bearing on the stop edge 20, is prevented from returning to the locking position.

For example, the actuation element 19, in the actuation position, can engage in a wedge-shaped depression 21 in the coupling portion 8 inserted into the receiving space 14. Accordingly, the actuation element 19 can be movable out of the depression 21 by moving back to the rest position. The depression 21 can be delimited, in a direction transverse to the separation direction D2, by two obliquely oriented boundary edges 22. The boundary edges 22 can be oriented with respect to each other such that they run toward each other when viewed in the direction of insertion D1.

The plug connection can be configured in such a way that a portion of the actuation element 19 engaging in the depression 21 is moved toward the boundary edges 22 upon movement of the coupling portion 8 in the separation direction D2 and, when the coupling portion 8 reaches the intermediate position, abuts on both sides at the boundary edges 22, such that further movement of the coupling portion 8 beyond the intermediate position is not possible. The plug connection can be fully disconnected only when the actuation element 19 is moved back out of the depression 21 such that the displacement path is released.

The actuation element 19 can be configured, for example, as an actuation tab 19. The actuation tab 19 can be connected at one of its ends to the coupling receptacle 3a, 3b, more exactly to an edge that delimits a through-opening 23 in a wall portion of the receiving space 14, and, by elastic bending of its other, unconnected end, can be movable into the actuation position, such that a spring force acts on the actuation tab 19 in the actuation position to return the actuation tab 19 to the rest position. When moving into the actuation position, the actuation tab 19 can be pivoted, for example, through the through-opening 23 into the interior of the receiving space 14.

In addition or alternatively, the locking element 18 can be configured as a locking tab 18. The locking tab 18 can be connected at one of its ends to the coupling portion 8, more exactly to an edge of the coupling portion 8 delimiting the depression 21, and, by elastic bending of its other, unconnected end, can be movable into the unlocking position, such that a spring force acts on the locking tab 18 in the unlocking position to return the locking tab 18 to the locking position. For example, when moving to the unlocking position, the locking tab 18 can be pivoted into the depression 21 together with the actuation tab 19 pressing against the locking tab 18.

The depression 21 can extend at least partially within a region of the coupling portion 8 surrounded by the outer contour 17.

It is possible that the locking tab 18 has, at its unconnected end, a projection 24 which in the locking position positively engages in the coupling receptacle 3a, 3b, for example in the through-opening 23, and which, in the unlocking position, releases the coupling receptacle 3a, 3b.

The actuation tab 19 can additionally have a specially structured, for example particularly ergonomic and/or particularly non-slip, touch surface 25 at which the actuation tab 19 can be pressed with a finger in order to unlock the plug connection. The touch surface 25 can for example be structured, preferably ribbed, in such a way that it is difficult for a finger pressing the touch surface 25 to slip in a direction parallel to the insertion direction D1 or to the separation direction D2.

In addition, the respiratory mask 1 can comprise a mask bead 26 for supporting the mask body 2 on the skin of the patient's face. Thus, the mask body 2 can be applied to the face via the mask bead 26. The mask bead 26 can be configured to be considerably more flexible than the mask body 2 and to connect the mask body 2 to the facial skin in an airtight manner during use of the respiratory mask 1.

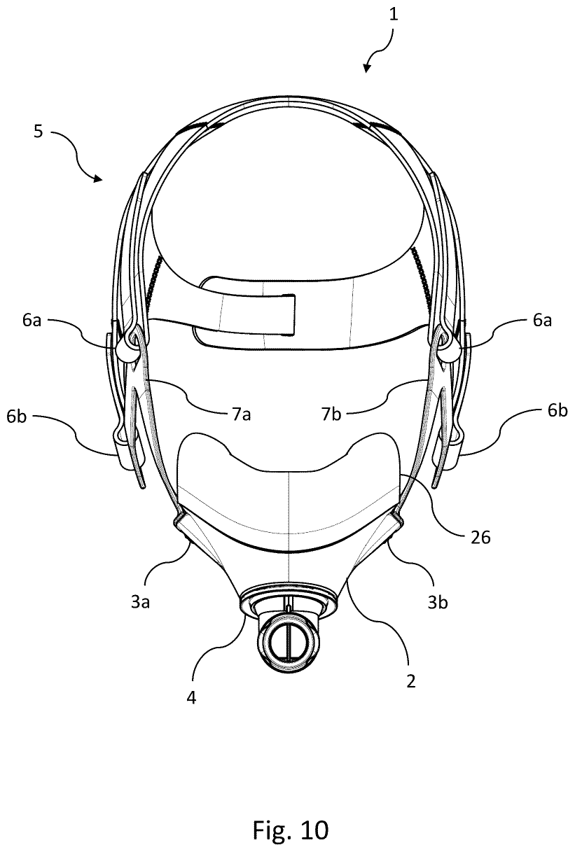

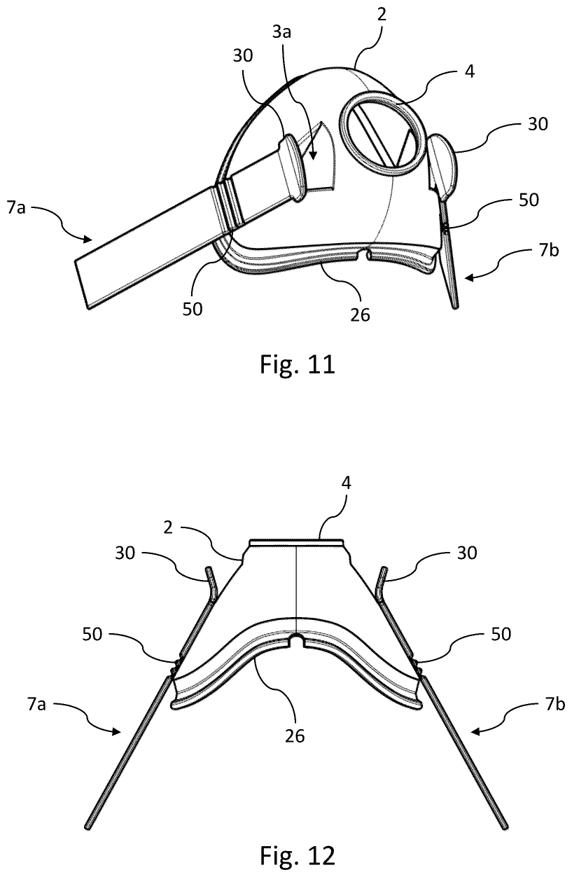

FIG. 11 shows a variant in which the mask body 2 can be releasably connected to the two coupling elements 7a, 7b via a hooking arrangement instead of via a plug connection.

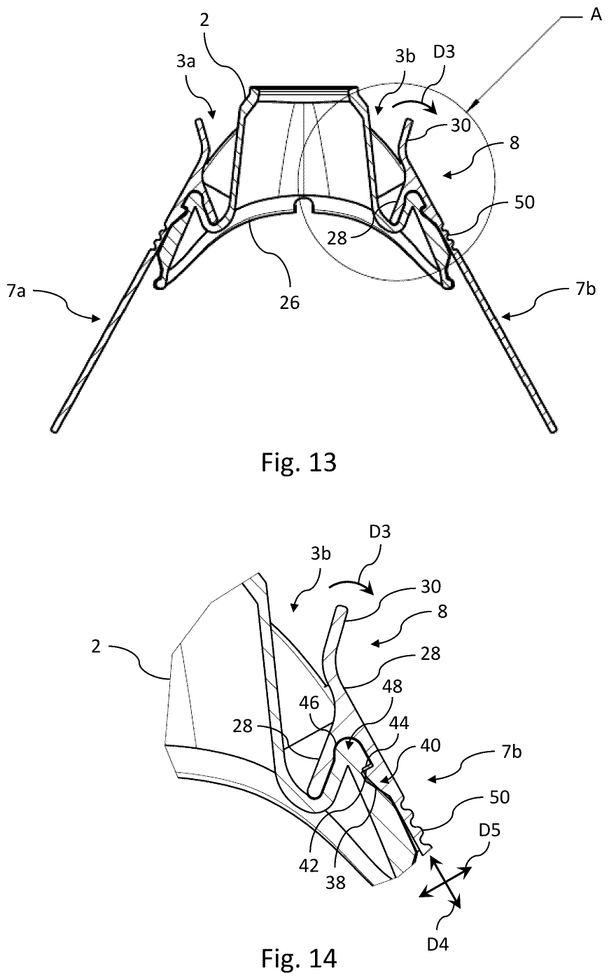

As shown in FIG. 13 to FIG. 15, in this example the coupling portion 8 of each coupling element 7a, 7b comprises a hook-shaped hook portion 28, which can be hooked onto the respective coupling receptacle 3a, 3b, in order to form a hooking arrangement for fastening the head harness 5 to the mask body 2. Furthermore, the coupling portion 8 of each coupling element 7a, 7b comprises a wing-shaped actuation portion 30 which is firmly connected to the hook portion 28 and, when the mask body 2 is fixed with respect to the patient's face by means of the head harness 5, protrudes from the rest of the coupling portion 8 in such a way that it can be pivoted with at least one finger in a pivoting direction D3 away from the respective coupling receptacle 3a, 3b, i.e. away from the mask body 2 in each case.

The hooking arrangement is also configured in such a way that, when the mask body 2 is fixed with respect to the patient's face by means of the head harness 5, the hook portion 28 is pressed against the respective coupling receptacle 3a, 3b by a tensile force exerted by the head harness 5. This can prevent unintentional release, for example accidental release, of the hooking arrangement. Furthermore, the hooking arrangement is configured in such a way that the hook portion 28 can be moved out of the respective coupling receptacle 3a, 3b by pivoting of the actuation portion 30 in the pivoting direction D3. This allows a controlled release of the hooking arrangement.

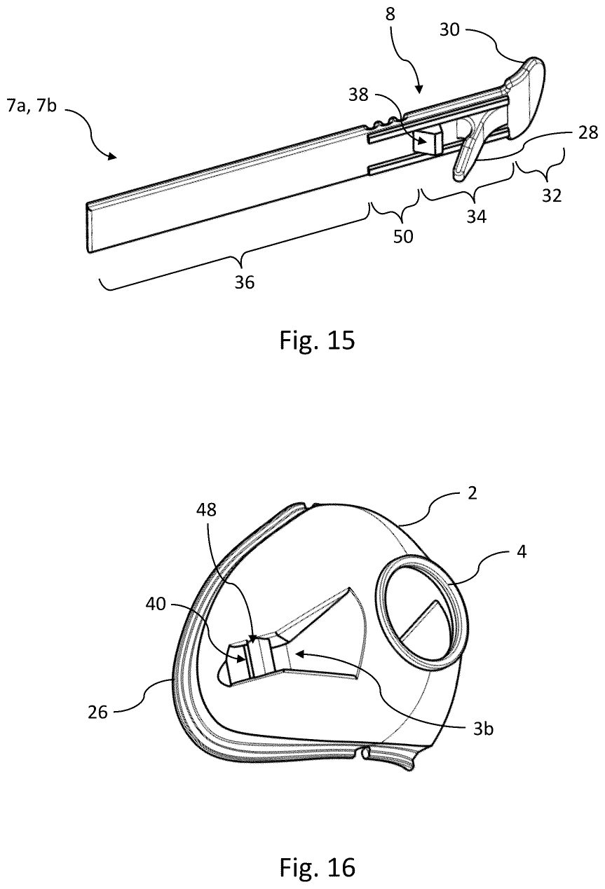

As can be easily seen in FIG. 15, the coupling element 7a, 7b, viewed in the longitudinal direction thereof, can be divided into at least a first longitudinal portion 32, a second longitudinal portion 34 and a third longitudinal portion 36. In this case, the second longitudinal portion 34 can be arranged between the first longitudinal portion 32 and the third longitudinal portion 36. In addition, the first longitudinal portion 32 can protrude obliquely or in a straight line from one end (here the end facing away from the third longitudinal portion 36) of the second longitudinal portion 34. This protruding part of the first longitudinal portion 32 can function as the actuation portion 30. Furthermore, the second longitudinal portion 34 can comprise the hook portion 28. The third longitudinal portion 36 can be a remaining longitudinal portion (of greater or lesser length) of the respective coupling element 7a, 7b, the end of which facing away from the second longitudinal portion 34 can be connected in a suitable manner to the respective strap of the head harness 5.

In addition, the coupling portion 8 of the respective coupling element 7a, 7b, for example its second longitudinal portion 34, can comprise a nose 38, which can be configured to engage in a recess 40, complementary to the nose 38, of the respective coupling receptacle 3a, 3b and/or of the mask body 2 when the hook portion 28 is hooked onto the respective coupling receptacle 3a, 3b. As is shown in FIG. 15, the hook portion 28 can, for example, be arranged at least partially between the nose 38 and the actuation portion 30, when viewed in the longitudinal direction of the respective coupling element 7a, 7b. In addition, the nose 38 and the hook portion 28 can each protrude from the same side of the respective coupling element 7a, 7b, wherein the hook portion 28 can partially or completely protrude past the nose 38.

As can be seen in FIG. 14, the nose 38 can have a stop surface 42 and is further configured such that the stop surface 42 of the nose 38 engaging in the recess 40, upon movement of the coupling portion 8 relative to the respective coupling receptacle 3a, 3b in at least one direction of movement D4, here parallel to an effective direction of the tensile force exerted by the head harness 5, strikes against a counter-surface 44 of the recess 40, such that further movement of the coupling portion 8 relative to the respective coupling receptacle 3a, 3b in the respective direction of movement D4 is not possible. Nevertheless, a movement of the coupling portion 8 relative to the respective coupling receptacle 3a, 3b in at least one further direction of movement D5 deviating from the direction of movement D4, here orthogonal to the direction of movement D4 (more precisely, a relative movement between the stop surface 42 and the counter-surface 44 touching the stop surface 42 in the further direction of movement D5), is still possible to a limited extent without the hooking arrangement being released.

The nose 38 can advantageously be wedge-shaped at least in sections. Upon controlled release of the hooking arrangement, this allows the nose 38 to slide out of the recess 40 solely due to the tensile force exerted by the head harness 5.

In addition, the hook portion 28 can have a rounded hook inner contour 46, which engages around a rounded coupling contour 48 of the respective coupling receptacle 3a, 3b, complementary to the hook inner contour 46, when the hook portion 28 is hooked onto the respective coupling receptacle 3a, 3b). The hooking arrangement can be configured in such a way that, when the mask body 2 is fixed with respect to the patient's face by means of the head harness 5, the hook inner contour 46 engaging around the coupling contour 48 is pressed against the coupling contour 48 by the tensile force exerted by the head harness 5, in order to prevent the unintentional release of the hooking arrangement, and, during the pivoting of the actuation portion 30 in the pivoting direction D3, is moved at least partially along the coupling contour 48, in order to enable a corresponding pivoting movement of the hook portion 28 out of the respective coupling receptacle 3a, 3b and thus the controlled release of the hooking arrangement.

In addition, the coupling portion 8 of the respective coupling element 7a, 7b, for example the second longitudinal portion 34, can be connected to a remaining longitudinal portion of the respective coupling element 7a, 7b, for example the third longitudinal portion 36, via a special bending portion 50. The bending portion 50 can be particularly flexible in comparison to the coupling portion 8 and to the remaining longitudinal portion. This allows the coupling portion 8 to be angled relative to the remaining longitudinal portion, which plays a role for example when using the respiratory mask 1 on faces having different widths. For example, a thickness of the bending portion 50, viewed in the longitudinal direction of the respective coupling element 7a, 7b, can vary between two different thickness values, according to a predetermined (here undulating or sinusoidal) thickness profile. This permits a particularly flexible and yet stable mechanical connection between the coupling portion 8 and the remaining longitudinal portion. In this case, the bending portion 50 can be made of the same material (for example the same preferably thermoplastic material) as the remaining coupling element 7a, 7b.

Finally, it will be noted that terms such as “have”, “comprise”, “include”, “with” etc. do not exclude other elements or steps, and indefinite articles such as “a” or “an” do not exclude a plurality.

Furthermore, it will be noted that features or steps described with reference to one of the embodiments above can also be used in combination with features or steps described with reference to other embodiments from among the embodiments above.

Reference signs in the claims should not be understood as restricting the scope of the subject matter defined by the claims.

List of reference numerals

| | |
|---|---|
| 1 | respiratory mask |
| 2 | mask body |
| 3a | first coupling receptacle |
| 3b | second coupling receptacle |
| 4 | tube connector |
| 5 | head harness |
| 6a | first strap |
| 6b | second strap |
| 7a | first coupling element |
| 7b | second coupling element |
| 8 | coupling portion |
| 9a | first strap receptacle |
| 9b | second strap receptacle |
| 10a | first arm |
| 10b | second arm |
| 11 | transition portion |
| 12 | recess opening |
| 13 | edge portion |
| 14 | receiving space |
| 15 | insertion opening |
| 16 | inner contour |
| 17 | outer contour |
| 18 | locking element, locking tab |
| 19 | actuation element, actuation tab |
| 20 | stop edge |
| 21 | depression |
| 22 | boundary edge |
| 23 | through-opening |
| 24 | projection |
| 25 | touch surface |
| 26 | mask bead |
| 28 | hook portion |
| 30 | actuation portion |
| 32 | first longitudinal portion |
| 34 | second longitudinal portion |
| 36 | third longitudinal portion |
| 38 | nose |
| 40 | recess |
| 42 | stop surface |
| 44 | counter-surface |
| 46 | hook inner contour |
| 48 | coupling contour |
| 50 | bending portion |
| D1 | insertion direction |
| D2 | separation direction |
| D3 | pivoting direction |
| D4 | direction of movement |
| D5 | further direction of movement |
| F1 | first tensile force |
| F2 | second tensile force |
| H | sagittal axis, horizontal |
| K | head |
| V | longitudinal axis, vertical |

What is claimed is:

1. A respiratory mask for supplying a patient with respiratory gas, wherein the respiratory mask comprises:
a mask body for applying to the mouth and/or nose of the patient, the mask body comprising a first coupling receptacle, a second coupling receptacle, and a tube connector for attachment of a breathing tube;
a head harness for fixing the mask body with respect to the patient's face, the head harness comprising a first strap and a second strap for tensioning around the patient's head;
a first coupling element for coupling the head harness to the first coupling receptacle;
a second coupling element for coupling the head harness to the second coupling receptacle;
wherein each coupling element comprises a coupling portion for coupling the coupling element to the respective coupling receptacle, a first strap receptacle for fastening the first strap (and a second strap receptacle for fastening the second strap;
and wherein each coupling element is configured such that, when the mask body is fixed with respect to the patient's face by means of the head harness, the first strap receptacle, viewed in a direction of a sagittal axis (H) of the patient, is arranged at least partially between a chin and an ear of the patient and, viewed in a direction of a longitudinal axis (V) of the patient orthogonal to the sagittal axis (H), is arranged at least partially between the chin and a temporal region of the patient, and the second strap receptacle is arranged at least partially opposite the temporal region of the patient.

2. The respiratory mask of claim 1,
wherein each coupling element is configured such that, when the mask body is fixed with respect to the patient's face by the head harness, the first strap receptacle is arranged at least partially opposite a part of a lower jaw of the patient.

3. The respiratory mask of claim 1,
wherein each coupling element is configured such that, when the mask body is fixed with respect to the patient's face by the head harness, the coupling portion coupled to the respective coupling receptacle, and viewed in a direction of the longitudinal axis (V), is arranged at least partially between the first strap receptacle and the second strap receptacle, and/or the second strap receptacle, viewed in a direction of the sagittal axis (H), is arranged at least partially between the first strap receptacle and the coupling portion coupled to the respective coupling receptacle.

4. The respiratory mask of claim 1,
wherein, when the mask body is fixed with respect to the patient's face by the head harness, the first strap exerts a first tensile force (F1) on the first strap receptacle of each coupling element and the second strap exerts a second tensile force (F2) on the second strap receptacle of each coupling element, wherein a component of the first tensile force (F1) acting in the direction of the sagittal axis (H) is greater than a component of the first tensile force (F1) acting in a direction of the longitudinal axis (V), and/or a component of the second tensile force (F2) acting in the direction of the sagittal axis (H) is greater than a component of the second tensile force (F2) acting in a direction of the longitudinal axis (V).

5. The respiratory mask of claim 1,
wherein each coupling element comprises a first arm and a second arm, wherein the first arm and the second arm are connected to each other in an L shape or V shape and transition into each other in a transition portion, wherein the first strap receptacle is arranged at least partially in the transition portion, wherein the second strap receptacle is arranged at an end of the second arm facing away from the transition portion, and wherein the coupling portion is arranged at an end of the first arm facing away from the transition portion.

6. The respiratory mask of claim 1,
wherein each first strap receptacle and/or each second strap receptacle comprises a receiving opening formed in the material of the respective coupling element, for insertion of the respective strap, wherein the receiving opening is at least partially bounded by an edge portion curved in a circumferential direction of the receiving opening, and wherein the strap fastened to the respective strap receptacle at least partially wraps around the edge portion, wherein an orientation of a longitudinal axis of the respective strap relative to the sagittal axis (H) can be varied by the part of the respective strap surrounding the edge portion being moved along the edge portion.

7. The respiratory mask of claim 1,
wherein a coupling portion of at least one of the coupling elements is connectable to the respective coupling receptacle to form a plug connection;
wherein the respiratory mask further comprises:
a locking element movably mounted on the mask body and/or on the respective coupling element between a locking position and an unlocking position, which locking element is configured to lock the plug connection in the locking position, so that a movement of the coupling portion in a separation direction (D2) away from the respective coupling receptacle is not possible, and to unlock it in the unlocking position;
an actuation element movably mounted on the mask body and/or on the respective coupling element between a rest position and an actuation position, for actuating the locking element when the plug connection is locked, and wherein the actuation element is configured to press against the locking element when moved to the actuation position, such that the locking element is moved to the unlocking position.

8. A respiratory mask for supplying a patient with respiratory gas, wherein the respiratory mask comprises:
a mask body for applying to a mouth and/or nose of the patient, the mask body comprising a first coupling receptacle, a second coupling receptacle, and a tube connector for attachment of a breathing tube;
a head harness for fixing the mask body with respect to the patient's face;
a first coupling element for coupling the head harness to the first coupling receptacle;
a second coupling element for coupling the head harness to the second coupling receptacle;
wherein each coupling element comprises a coupling portion for coupling the coupling element to the respective coupling receptacle, and a strap receptacle for fastening a strap of the head harness, wherein the coupling portion of at least one of the coupling elements is connectable to the respective coupling receptacle to form a plug connection;
and wherein the respiratory mask further comprises:
a locking element movably mounted on the mask body and/or on the respective coupling element between a locking position and an unlocking position, which locking element is configured to lock the plug connection in the locking position, so that a movement of the coupling portion in a separation direction (D2) away from the respective coupling receptacle is not possible, and to unlock it in the unlocking position;
an actuation element movably mounted on the mask body and/or on the respective coupling element between a rest position and an actuation position, for actuating the locking element when the plug connection is locked, the actuation element being configured to press against the locking element when moved to the actuation position, such that the locking element is moved to the unlocking position.

9. The respiratory mask of claim 8,
wherein the actuation element is configured to limit a movement path of the coupling portion during movement in the separation direction (D2) in the actuation position, such that the coupling portion can be moved only to an intermediate position relative to the respective coupling receptacle, and not to limit it in the rest position, such that the coupling portion can be moved beyond the intermediate position, the plug connection being able to be disconnected by moving the coupling portion beyond the intermediate position.

10. The respiratory mask of claim 9,
wherein the locking element, by movement of the coupling portion to the intermediate position, can be positioned opposite a stop edge of the mask body and/or of the respective coupling element, wherein the stop edge is configured to prevent the locking element positioned opposite the stop edge from returning to the locking position.

11. The respiratory mask of claim 9,
wherein the actuation element, in the actuation position, engages in a wedge-shaped depression in the mask body and/or in the respective coupling element and is movable out of the depression by moving back to the rest position, wherein the depression is bounded, in a direction transverse to the separation direction (D2), by two obliquely oriented boundary edges, wherein the actuation element is configured in such a way that a portion of the actuation element engaging in the depression is moved toward the boundary edges upon movement of the coupling portion in the separation direction (D2) and, when the coupling portion reaches an intermediate position, abuts on both sides at the boundary edges, such that further movement of the coupling portion beyond the intermediate position is not possible.

12. The respiratory mask of claim 8,
wherein the actuation element is configured as an actuation tab, wherein the actuation tab is connected at one of its ends to the mask body and/or the respective coupling element and, by elastic bending of its other end, is movable into the actuation position, such that a spring force acts on the actuation tab in the actuation position to return the actuation tab to the rest position; and/or
wherein the locking element is configured as a locking tab, wherein the locking tab is connected at one of its ends to the mask body and/or the respective coupling element and, by elastic bending of its other end, is movable into the unlocking position, such that a spring force acts on the locking tab in the unlocking position to return the locking tab to the locking position.

13. The respiratory mask of claim 1,
wherein the coupling portion of at least one of the coupling elements comprises:
a hook-shaped hook portion that can be hooked onto the respective coupling receptacle to form a hooking arrangement for fastening the head harness to the mask body;
a wing-shaped actuation portion which is firmly connected to the hook portion and, when the mask body is fixed with respect to a patient's face by the head harness, protrudes from a rest of the coupling portion in such a way that it can be pivoted with at least one finger in a pivoting direction (D3) away from the respective coupling receptacle;
wherein the hooking arrangement is configured in such a way that, when the mask body is fixed with respect to the patient's face by the head harness, the hook portion is pressed against the respective coupling receptacle by a tensile force exerted by the head harness, in order to prevent unintentional release of the hooking arrangement, and can be moved out of the respective coupling receptacle by pivoting of the actuation portion in the pivoting direction (D3), in order to enable a controlled release of the hooking arrangement.

14. A respiratory mask for supplying a patient with respiratory gas, wherein the respiratory mask comprises:
a mask body for applying to a mouth and/or nose of the patient, the mask body comprising a first coupling receptacle, a second coupling receptacle, and a tube connector for attachment of a breathing tube;
a head harness for fixing the mask body with respect to a patient's face;
a first coupling element for coupling the head harness to the first coupling receptacle;
a second coupling element for coupling the head harness to the second coupling receptacle;
wherein each coupling element comprises a coupling portion for coupling the coupling element to the respective coupling receptacle, and a strap receptacle for fastening a strap of the head harness;
wherein the coupling portion of at least one of the coupling elements comprises:
a hook-shaped hook portion that can be hooked onto the respective coupling receptacle to form a hooking arrangement for fastening the head harness to the mask body;
a wing-shaped actuation portion which is firmly connected to the hook portion and, when the mask body is fixed with respect to the patient's face by the head harness, protrudes from a rest of the coupling portion in such a way that it can be pivoted with at least one finger in a pivoting direction (D3) away from the respective coupling receptacle;
wherein the hooking arrangement is configured in such a way that, when the mask body is fixed with respect to the patient's face by the head harness, the hook portion is pressed against the respective coupling receptacle by a tensile force exerted by the head harness, in order to prevent unintentional release of the hooking arrangement, and can be moved out of the respective coupling receptacle by pivoting of the actuation portion in the pivoting direction (D3), in order to enable a controlled release of the hooking arrangement.

15. The respiratory mask of claim 14,
wherein the respective coupling element, viewed in its longitudinal direction, is divided into at least a first longitudinal portion, a second longitudinal portion and a third longitudinal portion, wherein the second longitudinal portion is arranged between the first longitudinal portion and the third longitudinal portion, wherein the first longitudinal portion protrudes from one end of the second longitudinal portion and comprises the actuation portion, wherein the second longitudinal portion comprises the hook portion, wherein the third longitudinal portion is a remaining longitudinal portion of the respective coupling element.

16. The respiratory mask of claim 14,
wherein the coupling portion of the respective coupling element further comprises:
a nose, which is configured to engage in a recess, complementary to the nose, of the respective coupling receptacle and/or of the mask body when the hook portion is hooked onto the respective coupling receptacle, wherein the nose has a stop surface and is further configured such that the stop surface of the nose engaging in the recess, upon movement of the coupling portion relative to the respective coupling receptacle in at least one direction of movement (D4) parallel to an effective direction of a tensile force exerted by the head harness, strikes against a counter-surface of the recess, such that further movement of the coupling portion relative to the respective coupling receptacle in the respective direction of movement (D4) is not possible, wherein a movement of the coupling portion relative to the respective coupling receptacle in at least one further direction of movement (D5) deviating from the direction of movement (D4) is still possible to a limited extent without the hooking arrangement being released.

17. The respiratory mask of claim 16,
wherein the nose is at least partially wedge-shaped, in order to allow the nose to slide out of the recess, when the hooking arrangement is released in a controlled manner, solely due to a tensile force exerted by the head harness.

18. The respiratory mask of claim 14,
wherein the hook portion has a rounded hook inner contour, which engages around a rounded coupling contour of the respective coupling receptacle, complementary to the hook inner contour, when the hook portion is hooked onto the respective coupling receptacle, wherein the hooking arrangement is configured in such a way that, when the mask body is fixed with respect to the patient's face by the head harness, the hook inner contour engaging around the coupling contour is pressed against the coupling contour by a tensile force exerted by the head harness, in order to prevent an unintentional release of the hooking arrangement, and, during a pivoting of the actuation portion in a pivoting direction (D3), is moved at least partially along the coupling contour, in order to enable a pivoting movement of the hook portion out of the respective coupling receptacle.

19. The respiratory mask of claim 1,
wherein the coupling portion of at least one of the coupling elements is connected to a remaining longitudinal portion of the respective coupling element via a bending portion, wherein the bending portion is flexible in comparison to the coupling portion and to the remaining longitudinal portion, in order to allow the coupling portion to be angled relative to the remaining longitudinal portion.

20. The respiratory mask of claim 8,
wherein the coupling portion of at least one of the coupling elements comprises:
a hook-shaped hook portion that can be hooked onto the respective coupling receptacle to form a hooking arrangement for fastening the head harness to the mask body;
a wing-shaped actuation portion which is firmly connected to the hook portion and, when the mask body is fixed with respect to a patient's face by the head harness, protrudes from a rest of the coupling portion in such a way that it can be pivoted with at least one finger in a pivoting direction (D3) away from the respective coupling receptacle;
wherein the hooking arrangement is configured in such a way that, when the mask body is fixed with respect to the patient's face by the head harness, the hook portion is pressed against the respective coupling receptacle by a tensile force exerted by the head harness, in order to prevent unintentional release of the hooking arrangement, and can be moved out of the respective coupling receptacle by pivoting of the actuation portion in the pivoting direction (D3), in order to enable a controlled release of the hooking arrangement.
